(19) United States
(12) Patent Application Publication
(10) Pub. No.: US 2011/0223150 A1
(43) Pub. Date: Sep. 15, 2011

(54) Neutraceutical-Based Topical Anxiolytic Agent and Method of Use

(76) Inventor: Garrett Blake HOLLOWAY, Kerrville, TX (US)

(21) Appl. No.: 12/949,746

(22) Filed: Nov. 18, 2010

Related U.S. Application Data
(60) Provisional application No. 61/262,534, filed on Nov. 18, 2009.

(51) Int. Cl. A61K 38/48; A61P 25/22

(52) U.S. Cl. 424/94.64

(57) ABSTRACT

A nutraceutical-based anxiolytic agent (composition) for topical application is described. The formulation utilizes a combination of active ingredients directed to up-regulate the parasympathetic nervous system and calming vagal nerve enervation and its resultant stress symptomatology. The active ingredients in the topical composition include GABA (gamma-aminobutyric acid), L-theanine, Phenibut (beta-phenyl-gamma-aminobutyric acid), and casein tryptic hydrolysase. The active ingredients are dissolved in a lecithin organogel carrier such as Lipoderm or Phloderm to provide a superior transdermal delivery system.

### CROSS REFERENCE TO RELATED APPLICATIONS

This application claims the benefit under Title 35 United States Code §119(e) of U.S. Provisional Patent Application Ser. No. 61/262,534 filed Nov. 18, 2009, the full disclosure of which is incorporated herein by reference.

### BACKGROUND OF THE INVENTION

1. Field of the Invention

The present invention relates generally to topical preparations for providing relief from symptoms of stress in individuals suffering from anxiety. The present invention relates more specifically to an improved nutraceutical transdermal topical agent for the amelioration and management of the physiological symptoms of anxiety.

2. Description of the Related Art

It is well known in the medical and dental fields to provide a variety of palliative agents to patients to reduce the generalized anxiety responses experienced by many individuals both before and during medical and dental procedures. Currently, such anxiety is addressed primarily by controlled class prescription medications. These commonly include sedatives, hypnotics, and antipsychotics which are administered orally or parenterally.

Less frequently, topical preparations are given for anxiety. The known topicals, however, tend to be less potent than the parenterals, have slower response times, shorter durations, and less precise dosage titration. Many of these drugs produce undesirable side effects and have the potential for tolerance and dependency. In many cases, these drugs are not well tolerated or produce excessive sedation.

It would therefore be beneficial to provide a nutraceutical-based topical agent suitable for use in the management of the physiological symptoms of anxiety. It would be helpful if such an agent would have a superior transdermal delivery system. It would further be beneficial if such an agent was easy to use and apply such that it could be dispensed in a metered dose pen or unit dose packets.

### SUMMARY OF THE INVENTION

In fulfillment of the above and other objectives the present invention provides a nutraceutical-based anxiolytic agent for topical application. The novel formulation utilizes a combination of active ingredients directed to up-regulate the parasympathetic nervous system and calming vagal nerve enervation and its resultant stress symptomatology. The active ingredients in the topical composition include GABA (gamma-aminobutyric acid), L-Theanine, Phenibut (beta-phenyl-gamma-aminobutyric acid), and Casein Tryptic Hydrolysase. The active ingredients are dissolved in a lecithin organogel carrier such as Lipoderm or Phloderm to provide a superior transdermal delivery system.

The resulting topical agent has many advantages over drugs currently used to reduce anxiety, especially during medical and dental procedures. The composition of the present invention is easily stored and packaged, has easy dosage titration and application, and provides non-invasive and non-agitative delivery. The formulation has a rapid response rate, low incidence of side effects, and low to no potential for development of dependency or tolerance. The resulting topical agent has application across a wide spectrum of anxiety based symptoms with relatively modest requirement for modification of the basic formulation for optimal application to a particular condition or symptom.

### BRIEF DESCRIPTION OF THE DRAWINGS

FIG. 1 is a flowchart of the method steps associated with the formulation of the anxiolytic agent for topical application of the present invention.

FIG. 2 is a flowchart of the method steps associated with the use of the anxiolytic agent for topical application of the present invention.

### DETAILED DESCRIPTION OF THE PREFERRED EMBODIMENT

As indicated above, the present invention relates primarily to a combination of several neutraceutical-based anxiolytic agents dissolved in a Lecithin organogel transdermal delivery system. This topical application is useful in the amelioration and management of the physiological symptoms of anxiety. The agent can be effective in relieving stress symptoms resulting from many conditions including generalized anxiety disorder, social anxiety disorder, post-traumatic stress disorder, medical and dental phobias, health procedure phobias, and panic disorders.

In one preferred embodiment of the present invention, a preferred quantity (by mass) of the active ingredients may be as follows: GABA A 200 mg, L-Theanine 200 mg, Phenibut 400 mg, and Casein Tryptic Hydrolysase 400 mg. The preferred topical carrier may be from the Lecithin organogels, e.g., Lipoderm or Phloderm.

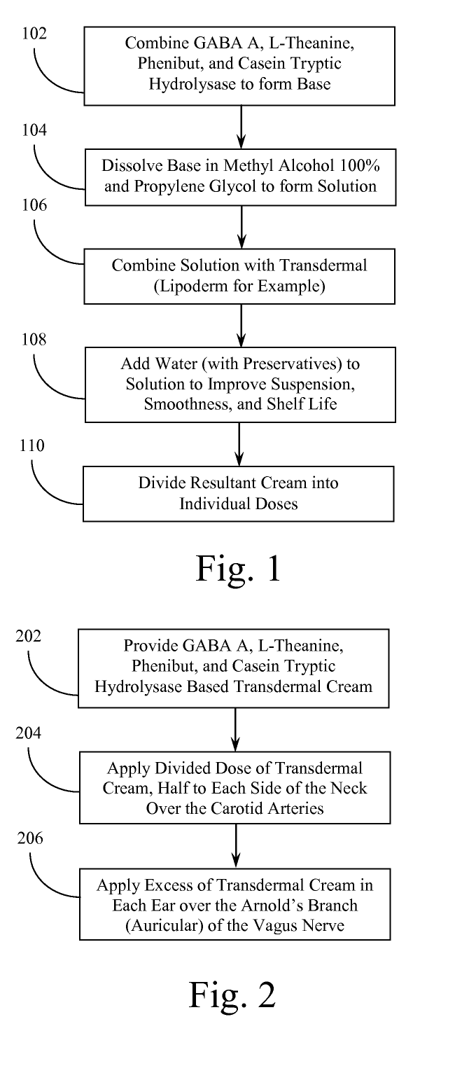

Reference is made to FIG. 1 wherein the laboratory, an effective compounded formula may be formulated as follows: GABA A 10 g, L-Theanine 10 g, Phenibut 20 g, and Casein Tryptic Hydrolysase 20 g combined (Step 102) and dissolved (Step 104) in 50 ml (40 ml ethyl alcohol 100% and 10 ml propylene glycol) to yield 1.2 g/ml. The transdermal may preferably have 3.5 g base per 35 g transdermal (Lipoderm) (Step 106). Water may be added to improve the suspension and smoothness of the compound (Step 108). Preservative may also be included to enhance the shelf life of the compound. The oral dose 100%=1200 mg of active ingredients. The transdermal bioavailability requires only 8%-10% of the oral dose=96 mg or 0.096 g (120 mg or 0.12 g). The transdermal dose is equivalent if not greater than oral delivery. The resultant cream may then be divided into individual doses (Step 110) for dispensing.

The compound may be delivered in a number of ways, a typical example of which is described in FIG. 2. Essentially the cream (Step 202) is dispensed to the neck over the left and right carotid arteries (Step 204) and also applied in the ear over the Arnold's Branch of the vagus nerve (Step 206). For example, the compound may be delivered by a dose metering pen wherein one click of the pen dispenses 0.05 g. Other ingredients may be added such as myoinositol, glycine, and N-acetyl L-tyrosine. Depending on the application and known interactions, the formula may be adjusted to include other ingredients for other applications such as lowering blood pressure, PTSD, and smoking cessation.

The pathways of action are the brain and the vagus nerve. The agent is applied topically to the area of the neck over the carotid arteries and to the area in each ear over Arnold's branch (auricular branch) of the vagus nerve. The application of the transdermal formula is preferably 1.2 g/ml which is applied in a divided dose, half to each side of the neck with the excess applied into each ear over Arnold's branch. Up to 50% more may be used for individuals weighing over 180 pounds. The agent may be delivered by a metered dose pen, unit dose packets, vial and non-needle syringe, or other convenient dosing method.

The initial onset of effect is within three minutes. The duration of effect is approximately 1½-2 hours. The desired effect reported is a sense of relaxation, calm, and greatly diminished anxiety. Infrequent side effects may include drowsiness or lightheadedness. The compound is stored at room temperature and has a shelf life of about 12-24 months. If water is included in the formulation, the compound will need to be refrigerated. Preservatives may be included to further enhance shelf life. The agent is generally contraindicated in pregnancy or those with a known allergy to any of the ingredients.

The compound is reported as well tolerated for all short term usages. For long term protocols such as smoking cessation, the compound may be produced in two formulations: one with Phenibut and one without Phenibut. This allows for a 3-5 day period for use of the formulation without Phenibut to prevent the development of tolerance. For other causes of anxiety, excipients may be added or removed to accommodate for the specific physiologic effects.

### Background on Composition Components

Gamma-aminobutyric acid (GABA) is a nonessential amino acid and important neurotransmitter. By decreasing the stimulation of neuronal activity it calms the body and mind and is often used as an addiction-free natural alternative to tranquilizers such as Valium and Librium. GABA also has reduced blood pressure in animal and human studies, boosted growth hormone production, successfully treated patients suffering from attention deficit disorder, seizures and strokes, and improved immunity. GABA may be referred to as a natural tranquilizer. Evidence suggests that major depression is accompanied by dysfunctional GABA systems. Abnormally low GABA concentrations were found in the brains of depressed patients (a highly significant 52% reduction) compared with healthy controls. This finding isn't surprising considering the anti-anxiety drugs classified as benzodiazepines-diazepam (Valium®), chlordiazepoxide (Librium®), and alprazolam (Xanax®) work by enhancing the action of GABA, which in turn prevents the neuronal agitation that causes anxiety.

Rather than taking the indirect route of trying to raise GABA levels through pharmaceutical intervention, a more direct approach, where GABA levels are elevated through supplementation, may be a superior alternative. Because GABA easily crosses the blood-brain barrier, it has been used in humans for its sedative and anesthetic properties. GABA is frequently given to produce a tranquilizing effect and to successfully treat patients suffering from anxiety or depression.

L-Theanine is a unique anxiety reducer and mood enhancer. Many studies have shown the health benefits of green tea, but what makes it the most consumed beverage in the world after water is its pleasant taste and relaxation effect. Both of these qualities can be traced to L-theanine (gamma-ethylamino-L-glutamic acid), a unique, neurologically-active amino acid. L-theanine is a free (non-protein) amino acid found almost exclusively in tea plants (Camellia sp.), constituting between 1% and 2% of the dry weight of tea leaves. It is the predominant amino acid in green tea leaves. Isolating L-theanine, with its physical and neurological benefits, from tea leaves was once difficult, expensive, and inefficient. Economically feasible methods of producing the identical L-theanine now exist and do not require large quantities of tea leaves.

Green tea's physiological effect of producing calm may seem contradictory to the stimulatory property of tea's caffeine content but this can be explained by the action of L-theanine This amino acid acts antagonistically against the stimulatory effects of caffeine on the nervous system. Research has demonstrated that L-theanine can creates a sense of relaxation in approximately 30-40 minutes after ingestion according to at least two different mechanisms. First, the amino acid directly stimulates the production of alpha brain waves, creating a state of deep relaxation and mental alertness similar to what is achieved through meditation. Second, L-theanine is involved in the formation of the inhibitory neurotransmitter, gamma amino butyric acid (GABA). GABA (as described above) influences the levels of two other neurotransmitters, dopamine and serotonin, producing the key relaxation effect.

L-theanine has additionally been shown to promote alpha brain wave activity. Alpha brain wave activity is present in wakefulness where there is a relaxed and effortless alertness and In contrast, beta brain wave activity is seen in highly stressful situations and where there is difficulty in mental concentration and focus. It is well known that alpha brain waves are generated during a relaxed state and therefore alpha waves are used as an index of relaxation. In one study of brain wave responses to L-theanine, brain wave topography showed that alpha waves were observed from the back to the top of a person's head (occipital and parietal regions of the brain) within approximately 40 minutes after the subjects had taken either 50 or 200 mg of L-theanine. In a separate study, the intensity of alpha waves were determined to be dose dependent (with a 200 mg dose showing a significant increase over controls) and detectable after 30 minutes.

L-theanine may also exert subtle changes in biochemistry comparable to reported effects of massage or a relaxing hot bath. L-theanine's effects are similar to those of adaptogens (natural herb products given to increase the body's resistance to stress, trauma, anxiety and fatigue) in that there is modulation in the balance of neurotransmitters. L-theanine has a significant effect on the release or reduction of neurotransmitters (as mentioned above) like dopamine and serotonin, resulting in improved memory and learning ability. L-theanine may also influence emotions due to its effects on the increased release of dopamine. L-theanine reduces brain serotonin concentration by either curtailing serotonin synthesis or increasing degradation in the brain. Whereas too little serotonin is associated with depression, increased levels of serotonin are associated with anxiety.

It is also known that the regulation of blood pressure is partly dependent upon catecholaminergic and serotonergic neurons in both the brain and the peripheral nervous system. Studies on spontaneously hypertensive rats (SHR) have shown a blood pressure lowering effect with L-theanine. The lowered blood pressure effect was dose-dependent with the highest test dose creating the most significant drop. L-glutamine was used as one of the controls in these studies. Although L-glutamine is similar in chemical structure to L-theanine, it did not exhibit an anti-hypertensive effect.

Other preliminary studies have reported that L-theanine has been found to increase the anti-tumor activity of some chemotherapeutic agents (doxorubicin and idarubicin) and to ameliorate some of the side effects of these drugs. It appears to increase the inhibitory concentration of these drugs in the tumor cells, although the mechanism is not known. At the same time, L-theanine decreased oxidative stress caused by these agents on the normal cells, possibly due to its mild antioxidant activity. In this regard, L-theanine has been shown to inhibit lipid peroxidation, catalyzed by copper, in low-density lipoprotein (LDL) in vitro.

L-theanine is generally considered safe. In 1964, the Japanese Ministry of Health and Welfare approved L-theanine for unlimited use in all foods, with the exception of infant foods. The intended use of L-theanine is that of a mental and physical relaxant that does not induce drowsiness. The FDA recommends a maximum dose of 1,200 mg per day, although the reason for this limit is not clear, due to its demonstrated safety. There are no known adverse reactions to L-theanine and no drug interactions have been reported. L-theanine is not affected by food and may be taken anytime, as needed.

Phenibut (beta-phenyl-gamma-aminobutyric acid, sometime spelled fenibut, originally known as phenigamma) is a derivative of the neurotransmitter GABA that crosses the blood-brain barrier. It was developed in Russia, where it has been used clinically since the 1960's for a range of purposes. Phenibut has both nootropic and anxiolytic (anxiety-reducing) properties, and it is commonly compared to diazepam (Valium®), baclofen, and piracetam, and it has similarities to and differences from all of these substances.

Structurally, phenibut is similar to GABA, baclofen (p-Cl-phenibut), and beta-phenylethylamine (PEA). GABA is, as described above the primary inhibitory neurotransmitter in the brain. The addition of the phenyl ring to GABA allows the compound to more easily cross the blood-brain barrier, but also changes its activity. Baclofen is a drug commonly used in studies on GABA(B) receptors, and also clinically used to treat severe spasticity of cerebral origin. PEA is a naturally occurring biogenic amine which is similar in structure to amphetamine, and like amphetamine, it is a stimulant that causes the release of dopamine, and also promotes anxiety in high enough amounts.

Phenibut is a GABA receptor agonist and also causes the release of GABA. Similar to baclofen, phenibut is an agonist at GABA(B) receptors, although it does have some effect on GABA(A) receptors as well. It is possible that phenibut has a higher activity at central GABA(B) receptors than peripheral ones. The role of the GABA(B) receptor is not well-established, although research in the last seven years has significantly increased understanding of the receptor. The most well-established role of GABA(B) receptors is inhibition of the release of some neurotransmitters, and it may also serve as a negative feedback mechanism for GABA release.

Because of the structural similarity to PEA, phenibut may share some similarities and differences with it. When phenibut is administered along with PEA, it antagonizes many of its effects, such as promotion of anxiety, promotion of seizures, and hyperthermia. This has led some to postulate that antagonism of PEA, rather than the GABA-mimetic activity, may be the important mechanism of action for the anxiolytic effect of phenibut. Phenibut also increases dopamine levels, and it has been postulated that the structural similarity to PEA may play a role in this effect. Phenibut has been found effective in anxiety reduction in many animal models of anxiety, although there is often dependence on study conditions. In cats classified as “anxious” or “passive,” phenibut has been shown to reduce the fear response and increased aggression in a confrontational situation, while it had no effect on aggressive cats. In normal cats, it led to “positive emotional symptoms”. In mice, phenibut has been shown to increase social behavior. In rats, phenibut has been shown to decrease some of the physiological responses to stress, including the elevation of glucocorticoid levels. Phenibut has also been reported to decrease the fear response caused by electrical stimulation and counteract the anxiogenic effect of the beta-carboline DMCM.

Phenibut has a mechanism of action similar to that of many drugs which are known to reduce anxiety in humans. Animal studies have compared the profile of phenibut to diazepam (Valium®), which has pronounced anxiolytic properties, and piracetam, which has weak anxiolytic properties. One study found phenibut had a tranquilizing effect similar to, but weaker than diazepam. It also caused sedation and muscle relaxation (whereas piracetam did not), but again these effects were weaker than those caused by diazepam. In Russia, phenibut is commonly used to treat many neuroses, including post-traumatic stress disorder, stuttering, and insomnia. In double blind placebo-controlled studies, phenibut has reportedly been found to improve intellectual function, improve physical strength, and reduce fatigue in neurotic and psychotic patients.

Phenibut has generally low acute toxicity. Reported LD50s (dose required to kill 50% of laboratory animals) are 900 mg/kg i.p. in mice, 700 mg/kg i.p. in rats, and 1000 mg/kg in rats (method of administration not given). Chronic administration of 50 mg/kg did not have teratogenic effects in rats. In clinical studies, no signs of toxicity have been reported, and side effects are few. Some report drowsiness, but this effect is not nearly as likely or severe as with benzodiazepines.

Drug interactions may occur when taking phenibut. It may decrease the threshold dose and potentiate certain actions of a drug. It amplifies some of the effects of anesthetics (ether, chloral hydrate, and barbiturates), diazepam, alcohol, and morphine; it would also presumably have an interaction with related drugs, such as other opiates and GHB. In contrast, taking phenibut with some other drugs, such as stimulants, will more than likely just blunt their effect.

Tolerance develops to many of the effects of phenibut, although it is reported that it does not develop to the nootropic effect. The first signs of tolerance may be seen within as little as five days. For this reason, it is commonly used for one to two week periods, or dosage is increased by 25%-30% after two weeks. This makes phenibut ideal for short periods of stress or anxiety, but not ideal for chronic use. It is possible that taking only one dose daily may partially reduce the development of tolerance.

Casein Hydrolysate is a preparation made from the milk protein casein, which is hydrolyzed to break it down into its constituent amino acids. Drinking warm milk is believed to provide a calming effect, perhaps as a reminiscence of early childhood. Recent studies have provided a scientific basis for this belief. When casein, the major milk protein, is hydrolysed, in a controlled manner, into small peptides and screened for anxiolytic activity, one of the peptides is strongly apparent. The active decapeptide was isolated and its spatial structure, as determined through NMR spectroscopy and molecular dynamic simulation, reveals a similarity to that of benzodiazepines. The peptide was shown to bind GABA receptors in test tube assays. Animal studies also confirm that the decapeptide has in vivo anxiolytic properties.

The ingredient list of the current base includes: deionized water, medium chain triglicerides, simugel 600, GABA, lecithin, alcohol, glycerin L-theanine, sodium hydroxymethylglycinate, potassium sorbate and citric acid.

Of the specialty emulsifiers and gelling agents, Simugel 600 may preferably be used in the current formula. Simugel 600 is a thickening and emulsifying polymer for making emulsions or for thickening aqueous and solvent based systems. Simulgel EG is an acrylic co-polymer used to form gels of high stability without neutralization. It is especially useful for thickening solvents such as ethanol, hexylene glycol, propylene glycol or glycerin. Sepigel 305 a thickening and stabilizing agent for emulsions and gels in a ready to use emulsion. Simulgel NS is a new version of 305 with a lighter feel. Capigel 98 is a thickening and gelling agent for water based systems and surfactant or ionic based products. Sepiplus 265 and 400 are similar to the Sepigel 305 or Simulgel NS but are more salt tolerant.

The emulsifier Montanov 68® is an alkyl polyglucoside emulsifier made from fructose and natural alcohols from coconut oil. In Montanov 68, C16-C18 alcohols are used. It is unique in that it makes very small liquid crystal emulsifications. Montanov 68 will easily handle high percentages of silicones and create a stable emulsion. Montanov L® is a new emulsifier that only makes milks and light lotions with low viscosity. Monatanov 14 is used in combination with Montanov 68 or Sepigel 305 to give various feels to the emulsions.

Lecithin organogels are used as a potential phospholipid-structured system for topical drug delivery. As discussed in one study, lecithin organogels are clear, thermodynamically stable, viscoelastic and biocompatible jelly-like phases, chiefly comprised of hydrated phospholipids and appropriate organic liquid. A number of therapeutic agents have been formulated as lecithin organogels for their facilitated transport through topical route (for dermal or transdermal effect). The improved topical drug delivery has mainly been attributed to the biphasic drug solubility, the desired drug partitioning, and the modification of skin barrier function by the organogel components. Being thermodynamically stable, lecithin organogels are prepared by spontaneous emulsification and therefore possess prolonged shelf life. The utility of this novel matrix as a topical vehicle is furthered by its very low skin irritancy potential.

### Effective Ranges of Composition Constituents

Although variations in the relative quantities of the compound's primary constituents have been show to retain efficacy, there are preferred ranges set forth in Table I below that provide general guidance for the production of an effective formulation of the compound of the present invention and include the sample described above.

TABLE I

| | | Sample | Mass in | Percent of | Range |
|---|---|---|---|---|---|
| Constituent | a/k/a | Mass (g) | Base (g) | Cream (%) | (%) |
| GABA | gamma- | 10.0 | 6.6/200 | 3.33 | 1-5 |
| | aminobutyric acid | | | | |
| L-Theanine | L-gamma- | 10.0 | 6.6/200 | 3.33 | 1-5 |
| | glutamylethylamide | | | | |
| Phenibut | beta-phenyl- | 20.0 | 13.2/200 | 6.66 | 3-10 |
| | gamma- | | | | |
| | aminobutyric acid | | | | |
| Casein | Casein | 20.0 | 13.2/200 | 6.66 | 3-10 |
| Tryptic | | | | | |
| Hydrolase | | | | | |

The result of the Sample (described above and repeated in Table I) produces 60.0 g of base, 40.0 g of which will be added into the secondary constituents as described above and repeated in Table II. This 40/60 or 66% amount of the mixture is incorporated into Lipoderm (as an example) and the other constituents to make 200 ml of a 20% strength cream. Table II provides sample inactive constituents quantities into which the 40.0 g quantity of the base (the above composition) may be dissolved according to the examples provided in the text above.

TABLE II

| | | | Percent of | |
|---|---|---|---|---|
| Constituent | Sample | Ratio in Solution | Solution (%) | Range (%) |
| (preserved) | 40 ml | 40/200 | 20 | 5-40 |
| Propylene Glycol | 50 ml | 50/200 | 25 | 10-50 |
| Emulsifix-205 | 3.5 | 50/200 | 25 | 10-50 |
| Lipoderm | 83.5 | 83.5/200 | 41.75 | 30-60 |

Use preserved with methylparabens and propylparabens or similar preservatives sufficient to provide a stable shelf life of 3 months or more in cool or refrigerated conditions. Include in patent. For a 10%-40% base formula mix powders with liquid ingredients using standard compounding procedures. Mill final cream until it reaches mixture Level 1. Dispense into divided doses of 1.5 ml.

For a base formula example of 20% mix 40 g base in glass mortar with preserved. Add propylene glycol and Emulsifix 205 to the mixture and thoroughly mix. Gradually stir with Lipoderm to 200 ml. Mixing very thoroughly using mortar, mix and/or electronic mixer. After mixing, run through an ointment mill starting at Level 3 and finishing at Level 1 setting. Make as many runs as necessary to thoroughly mix and mill.

The preferred embodiment of the present invention maintains the percentage of the four major ingredients the same and the strength at 20%. The problem of a possible flaking of the drying cream flaking is related to not only the amount of active ingredients needed for the effect desired but the protocol of dissolving it and keeping it in suspension as described above. The mill and processing of the completed cream as described above is but one example of such appropriate for the present invention. The sample formula described above results in a cream that is very smooth, does not flake, and is concentrated. Lipoderm is effective at only 5% concentration so it is anticipated that one may be able to use less and substitute other cream substances without losing effectiveness.

Although the present invention has been described in terms of the foregoing preferred embodiments, this description has been provided by way of explanation only, and is not intended to be construed as a limitation of the invention. Those skilled in the art will recognize modifications in the present invention that might accommodate specific medical or dental environments. Such modifications do not necessarily depart from the spirit and scope of the invention.

I Claim:

1. An anxiolytic, transdermal, topical composition for the amelioration and management of the physiological symptoms of anxiety, wherein the composition is a mixture comprising:
(a) a pharmacologically active quantity of GABA;
(b) a pharmacologically active quantity of L-Theanine;
(c) a pharmacologically active quantity of Phenibut;
(d) a pharmacologically active quantity of Casein tryptic hydrolysase; and
(e) a topical transdermal carrier.

2. A method for the amelioration and management of the physiological symptoms of anxiety, the method comprising topically administering a quantity of a composition comprising a mixture of:
(a) a pharmacologically active quantity of GABA;
(b) a pharmacologically active quantity of L-Theanine;
(c) a pharmacologically active quantity of Phenibut;
(d) a pharmacologically active quantity of Casein tryptic hydrolysase; and
(e) a topical transdermal carrier.
